SEQUENCE LISTING (161 sequences)

SEQ ID NO: 1 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 2 - protein, 108 aa, Mus musculus
ENVLTQSPAIMAASLGQKVTMTCSASSSVSSSYLHWYQQKSGASPKPLIHRTSNLASGVPVRFSGSGSGTSYSLTISSVEAEDDATYYCQQWSGYPFTFGAGTKLELK

SEQ ID NO: 3 - protein, 111 aa, Mus musculus
DIVLTQSPASLAVSLGQRATISCRASESVDDYGISFMNWFQQKPGQPPKLLIYAASNQGSGVPARFSGSGSGTDFSLNIHPMEEDDTAMYFCQQSKEVPWTFGGGTRLEIK

SEQ ID NO: 4 - protein, 107 aa, Mus musculus
DIQMTQTTSSLSASLGDRVTINCRASQDINNYLNWYQQKPDGTVKLLIYYTSRLHSGVPSRFSGSGSGTDYSLTISSLEQEDFATYFCQQGDTLPRTFGGGTKLEIK

SEQ ID NO: 5 - protein, 108 aa, Mus musculus
ENVLTQSPAIMAASLGQKVTMTCSASSSVSSSYLHWYQQKSGASPKPLIHRTSNLASGVPARFSGSGSGTSYSLTISSVEAEDDATYFCQQWSGYPLTFGAGTKLELK

SEQ ID NO: 6 - protein, 17 aa, Mus musculus
LNWYQQKPDGTVKLLIY

SEQ ID NO: 7 - protein, 17 aa, Mus musculus
LHWYQQKSGASPKPLIH

SEQ ID NO: 8 - protein, 17 aa, Mus musculus
MNWFQQKPGQPPKLLIY

SEQ ID NO: 9 - protein, 17 aa, Mus musculus
LNWYQQKPDGTVKLLIY

SEQ ID NO: 10 - protein, 17 aa, Mus musculus
LHWYQQKSGASPKPLIH

SEQ ID NO: 11 - protein, 36 aa, Mus musculus
RLHSGVPSRFSGSGSGTDYSLTISNLEQEDIATYFC

SEQ ID NO: 12 - protein, 36 aa, Mus musculus
NLASGVPVRFSGSGSGTSYSLTISSVEAEDDATYYC

SEQ ID NO: 13 - protein, 36 aa, Mus musculus
NQGSGVPARFSGSGSGTDFSLNIHPMEEDDTAMYFC

SEQ ID NO: 14 - protein, 36 aa, Mus musculus
RLHSGVPSRFSGSGSGTDYSLTISSLEQEDFATYFC

SEQ ID NO: 15 - protein, 36 aa, Mus musculus
NLASGVPARFSGSGSGTSYSLTISSVEAEDDATYFC

SEQ ID NO: 16 - protein, 36 aa, Mus musculus; identical to SEQ ID NO: 11

SEQ ID NO: 17 - protein, 36 aa, Mus musculus; identical to SEQ ID NO: 12

SEQ ID NO: 18 - protein, 36 aa, Mus musculus; identical to SEQ ID NO: 13

SEQ ID NO: 19 - protein, 36 aa, Mus musculus; identical to SEQ ID NO: 14

SEQ ID NO: 20 - protein, 36 aa, Mus musculus; identical to SEQ ID NO: 15

SEQ ID NO: 21 - protein, 10 aa, Mus musculus
FGGGTKLEIK

SEQ ID NO: 22 - protein, 10 aa, Mus musculus
FGAGTKLELK

SEQ ID NO: 23 - protein, 10 aa, Mus musculus
FGGGTRLEIK

SEQ ID NO: 24 - protein, 10 aa, Mus musculus
FGGGTKLEIK

SEQ ID NO: 25 - protein, 10 aa, Mus musculus
FGAGTKLELK

SEQ ID NO: 26 - protein, 6 aa, Mus musculus
QDISNY

SEQ ID NO: 27 - protein, 7 aa, Mus musculus
SSVSSSY

SEQ ID NO: 28 - protein, 10 aa, Mus musculus
ESVDDYGISF

SEQ ID NO: 29 - protein, 6 aa, Mus musculus
QDINNY

SEQ ID NO: 30 - protein, 7 aa, Mus musculus
SSVSSSY

SEQ ID NO: 31 - protein, 3 aa, Mus musculus
YTS

SEQ ID NO: 32 - protein, 3 aa, Mus musculus
RTS

SEQ ID NO: 33 - protein, 3 aa, Mus musculus
AAS

SEQ ID NO: 34 - protein, 3 aa, Mus musculus
YTS

SEQ ID NO: 35 - protein, 3 aa, Mus musculus
RTS

SEQ ID NO: 36 - protein, 9 aa, Mus musculus
QQGNTLPWT

SEQ ID NO: 37 - protein, 9 aa, Mus musculus
QQWSGYPFT

SEQ ID NO: 38 - protein, 9 aa, Mus musculus
QQSKEVPWT

SEQ ID NO: 39 - protein, 9 aa, Mus musculus
QQGDTLPRT

SEQ ID NO: 40 - protein, 9 aa, Mus musculus
QQWSGYPLT

SEQ ID NO: 41 - protein, 123 aa, Mus musculus
QVQLQQSGPELVKPGASVKISCKASGYSFTSYYIYWVKQRPGQGLEWIGWIYPGSGNTKYNEKFKGKATLTADTSSSTAYMQLSSLTSEDSAVYYCARWSYYGSSYNYAMDYWGQGTSVTVSS

SEQ ID NO: 42 - protein, 116 aa, Mus musculus
QVQLQQSGAELVKPGASVKISCKASGYAFSSYWMNWVKQRPGKGLEWIGQIYPGNGDSKYNGKFKGKATLTADKSSSTAYMLLSSLTSEDSAVYFCARWEFRFAYWGQGTLVTVSA

SEQ ID NO: 43 - protein, 117 aa, Mus musculus
QVQLQQPGAELVKPGASVKMSCKASGYTFTSYWITWVKQRPGQGLEWIGDIYPGSGSTNYNEKFKSKATLTVDTSSSTAYMQLSSLTSEDSAVYYCARDNSYAMDYWGQGTSVTVSS

SEQ ID NO: 44 - protein, 118 aa, Mus musculus
QIQLQQSGPELVKPGASVKISCKASGYTFTDYYINWVKQRPGQGLEWIGWIYPGSGNTKYNEKFKGKATLTVDTSSSTAYMQLSSLTSEDSAVYFCARSYRGYAMDYWGQGTSVTVSS

SEQ ID NO: 45 - protein, 123 aa, Mus musculus
QVQLQQPGAELVKPGASVKLSCKASGYTFTSYWMHWVKQRPGRGLEWIGRIDPNSGGTKYNEKFKTKATLTVDKPSSTAYMQLSSLTSEDSAVYYCAREDYYGNYFYWYFDVWGTGTTVTVSS

SEQ ID NO: 46 - protein, 25 aa, Mus musculus
QVQLQQSGPELVKPGASVKISCKAS

SEQ ID NO: 47 - protein, 25 aa, Mus musculus
QVQLQQSGAELVKPGASVKISCKAS

SEQ ID NO: 48 - protein, 25 aa, Mus musculus [sequence omitted]

SEQ ID NO: 49 - protein, 25 aa, Mus musculus
QIQLQQSGPELVKPGASVKISCKAS

SEQ ID NO: 50 - protein, 25 aa, Mus musculus
QVQLQQPGAELVKPGASVKLSCKAS

SEQ ID NO: 51 - protein, 17 aa, Mus musculus
IYWVKQRPGQGLEWIGW

SEQ ID NO: 52 - protein, 17 aa, Mus musculus
MNWVKQRPGKGLEWIGQ

SEQ ID NO: 53 - protein, 17 aa, Mus musculus
ITWVKQRPGQGLEWIGD

SEQ ID NO: 54 - protein, 17 aa, Mus musculus
INWVKQRPGQGLEWIGW

SEQ ID NO: 55 - protein, 17 aa, Mus musculus
MHWVKQRPGRGLEWIGR

SEQ ID NO: 56 - protein, 38 aa, Mus musculus
KYNEKFKGKATLTADTSSSTAYMQLSSLTSEDSAVYYC

SEQ ID NO: 57 - protein, 38 aa, Mus musculus
KYNGKFKGKATLTADKSSSTAYMLLSSLTSEDSAVYFC

SEQ ID NO: 58 - protein, 38 aa, Mus musculus
NYNEKFKSKATLTVDTSSSTAYMQLSSLTSEDSAVYYC

SEQ ID NO: 59 - protein, 38 aa, Mus musculus
KYNEKFKGKATLTVDTSSSTAYMQLSSLTSEDSAVYFC

SEQ ID NO: 60 - protein, 38 aa, Mus musculus
KYNEKFKTKATLTVDKPSSTAYMQLSSLTSEDSAVYYC

SEQ ID NO: 61 - protein, 11 aa, Mus musculus
WGQGTSVTVSS

SEQ ID NO: 62 - protein, 11 aa, Mus musculus
WGQGTLVTVSA

SEQ ID NO: 63 - protein, 11 aa, Mus musculus
WGQGTSVTVSS

SEQ ID NO: 64 - protein, 11 aa, Mus musculus
WGQGTSVTVSS

SEQ ID NO: 65 - protein, 11 aa, Mus musculus
WGTGTTVTVSS

SEQ ID NO: 66 - protein, 8 aa, Mus musculus
GYSFTSYY

SEQ ID NO: 67 - protein, 8 aa, Mus musculus
GYAFSSYW

SEQ ID NO: 68 - protein, 8 aa, Mus musculus
GYTFTSYW

SEQ ID NO: 69 - protein, 8 aa, Mus musculus
GYTFTDYY

SEQ ID NO: 70 - protein, 8 aa, Mus musculus
GYTFTSYW

SEQ ID NO: 71 - protein, 8 aa, Mus musculus
IYPGSGNT

SEQ ID NO: 72 - protein, 8 aa, Mus musculus
IYPGNGDS

SEQ ID NO: 73 - protein, 8 aa, Mus musculus
IYPGSGST

SEQ ID NO: 74 - protein, 8 aa, Mus musculus
IYPGSGNT

SEQ ID NO: 75 - protein, 8 aa, Mus musculus
IDPNSGGT

SEQ ID NO: 76 - protein, 16 aa, Mus musculus
ARWSYYGSSYNYAMDY

SEQ ID NO: 77 - protein, 9 aa, Mus musculus
ARWEFRFAY

SEQ ID NO: 78 - protein, 10 aa, Mus musculus
ARDNSYAMDY

SEQ ID NO: 79 - protein, 11 aa, Mus musculus
ARSYRGYAMDY

SEQ ID NO: 80 - protein, 16 aa, Mus musculus
AREDYYGNYFYWYFDV

SEQ ID NO: 81 - DNA, 321 bp, Mus musculus
gatatccagatgacacagactacatcctccctgtctgcctctctgggagacagagtcaccatcagttgcagggcaagtcaggacatcagcaattatttaaactggtatcagcagaaaccagatggaactgttaaactcctgatctactacacatcaagattacactcaggagtcccatcaaggttcagtggcagtgggtctggaacagattattctctcaccattagcaacctggaacaagaagatattgccacttacttttgccaacagggtaatacgcttccgtggacgttcggtggaggcaccaagctggaaatcaaa

SEQ ID NO: 82 - DNA, 324 bp, Mus musculus
gaaaatgtgctcacccagtctccagcaataatggctgcctctctggggcagaaggtcaccatgacctgcagtgccagctcaagtgtaagttccagttacttgcactggtaccagcagaagtcaggcgcttcccccaaacccttgattcataggacatccaacctggcttctggagtcccagttcgcttcagtggcagtgggtctgggacctcttactctctcacaatcagcagcgtggaggctgaagatgatgcaacttattactgccagcagtggagtggttacccattcacgttcggtgctgggaccaagctggagctgaaa

SEQ ID NO: 83 - DNA, 333 bp, Mus musculus
gacattgtgctgacccaatctccagcttctttggctgtgtctctagggcagagggccaccatctcctgcagagccagcgaaagtgttgatgattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaggctggaaatcaaa

SEQ ID NO: 84 - DNA, 321 bp, Mus musculus
gatatccagatgacacagactacatcctccctgtctgcctctctgggagacagagtcaccatcaattgcagggcaagtcaggacattaacaattatttaaactggtatcagcagaaaccagatggaactgttaaactcctgatctactacacatcaagattacactcaggagtcccatcaaggttcagtggcagtgggtctggaacagattattctctcaccattagcagcctggaacaagaagattttgccacttacttttgccaacagggtgatacgcttcctcggacgttcggtggaggcaccaagttggaaatcaaa

SEQ ID NO: 85 - DNA, 324 bp, Mus musculus
gaaaatgtgctcacccagtctccagcaataatggctgcctctctggggcagaaggtcaccatgacctgcagtgccagctcaagtgtaagttccagttacttgcactggtaccagcagaagtcaggcgcttcccccaaacccttgattcataggacatccaacctggcttctggagtcccagctcgcttcagtggcagtgggtctgggacctcttactctctcacaatcagcagcgtggaggctgaagatgatgcaacttatttctgccagcagtggagtggttacccgctcacgttcggtgctgggaccaagctggagctgaaa

SEQ ID NO: 86 - DNA, 78 bp, Mus musculus
gatatccagatgacacagactacatcctccctgtctgcctctctgggagacagagtcaccatcagttgcagggcaagt

SEQ ID NO: 87 - DNA, 78 bp, Mus musculus
gaaaatgtgctcacccagtctccagcaataatggctgcctctctggggcagaaggtcaccatgacctgcagtgccagc

SEQ ID NO: 88 - DNA, 78 bp, Mus musculus
gacattgtgctgacccaatctccagcttctttggctgtgtctctagggcagagggccaccatctcctgcagagccagc

SEQ ID NO: 89 - DNA, 78 bp, Mus musculus
gatatccagatgacacagactacatcctccctgtctgcctctctgggagacagagtcaccatcaattgcagggcaagt

SEQ ID NO: 90 - DNA, 78 bp, Mus musculus; identical to SEQ ID NO: 87

SEQ ID NO: 91 - DNA, 51 bp, Mus musculus
ttaaactggtatcagcagaaaccagatggaactgttaaactcctgatctac

SEQ ID NO: 92 - DNA, 51 bp, Mus musculus
ttgcactggtaccagcagaagtcaggcgcttcccccaaacccttgattcat

SEQ ID NO: 93 - DNA, 51 bp, Mus musculus
atgaactggttccaacagaaaccaggacagccacccaaactcctcatctat

SEQ ID NO: 94 - DNA, 51 bp, Mus musculus; identical to SEQ ID NO: 91

SEQ ID NO: 95 - DNA, 51 bp, Mus musculus; identical to SEQ ID NO: 92

SEQ ID NO: 96 - DNA, 108 bp, Mus musculus
agattacactcaggagtcccatcaaggttcagtggcagtgggtctggaacagattattctctcaccattagcaacctggaacaagaagatattgccacttacttttgc

SEQ ID NO: 97 - DNA, 108 bp, Mus musculus
aacctggcttctggagtcccagttcgcttcagtggcagtgggtctgggacctcttactctctcacaatcagcagcgtggaggctgaagatgatgcaacttattactgc

SEQ ID NO: 98 - DNA, 108 bp, Mus musculus
aaccaaggatccggggtccctgccaggtttagtggcagtgggtctgggacagacttcagcctcaacatccatcctatggaggaggatgatactgcaatgtatttctgt

SEQ ID NO: 99 - DNA, 108 bp, Mus musculus
agattacactcaggagtcccatcaaggttcagtggcagtgggtctggaacagattattctctcaccattagcagcctggaacaagaagattttgccacttacttttgc

SEQ ID NO: 100 - DNA, 108 bp, Mus musculus
aacctggcttctggagtcccagctcgcttcagtggcagtgggtctgggacctcttactctctcacaatcagcagcgtggaggctgaagatgatgcaacttatttctgc

SEQ ID NO: 101 - DNA, 30 bp, Mus musculus
ttcggtggaggcaccaagctggaaatcaaa

SEQ ID NO: 102 - DNA, 30 bp, Mus musculus
ttcggtgctgggaccaagctggagctgaaa

SEQ ID NO: 103 - DNA, 30 bp, Mus musculus
ttcggtggaggcaccaggctggaaatcaaa

SEQ ID NO: 104 - DNA, 30 bp, Mus musculus
ttcggtggaggcaccaagttggaaatcaaa

SEQ ID NO: 105 - DNA, 30 bp, Mus musculus; identical to SEQ ID NO: 102

SEQ ID NO: 106 - DNA, 18 bp, Mus musculus
caggacatcagcaattat

SEQ ID NO: 107 - DNA, 21 bp, Mus musculus
tcaagtgtaagttccagttac

SEQ ID NO: 108 - DNA, 30 bp, Mus musculus
gaaagtgttgatgattatggcattagtttt

SEQ ID NO: 109 - DNA, 18 bp, Mus musculus
caggacattaacaattat

SEQ ID NO: 110 - DNA, 21 bp, Mus musculus
tcaagtgtaagttccagttac

SEQ ID NO: 111 - DNA, 9 bp, Mus musculus
tacacatca

SEQ ID NO: 112 - DNA, 9 bp, Mus musculus
aggacatcc

SEQ ID NO: 113 - DNA, 9 bp, Mus musculus
gctgcatcc

SEQ ID NO: 114 - DNA, 9 bp, Mus musculus
tacacatca

SEQ ID NO: 115 - DNA, 9 bp, Mus musculus
aggacatcc

SEQ ID NO: 116 - DNA, 27 bp, Mus musculus
caacagggtaatacgcttccgtggacg

SEQ ID NO: 117 - DNA, 27 bp, Mus musculus
cagcagtggagtggttacccattcacg

SEQ ID NO: 118 - DNA, 27 bp, Mus musculus
cagcaaagtaaggaggttccgtggacg

SEQ ID NO: 119 - DNA, 27 bp, Mus musculus
caacagggtgatacgcttcctcggacg

SEQ ID NO: 120 - DNA, 27 bp, Mus musculus
cagcagtggagtggttacccgctcacg

SEQ ID NO: 121 - DNA, 369 bp, Mus musculus
caggtccagctgcagcagtctggacctgagctggtgaagcctggggcttcagtgaagatatcctgcaaggcttctggctacagcttcacaagctactatatatattgggtgaagcagaggcctggacagggacttgagtggattggatggatttatcctggaagtggtaatactaagtacaatgagaagttcaagggcaaggccacactgacggcagacacatcctccagcactgcctacatgcagctcagcagcctaacatctgaggactctgcggtctattactgtgcaagatggtcttattacggtagtagctacaactatgctatggactactggggtcaaggaacctcagtcaccgtctcctca

SEQ ID NO: 122 - DNA, 348 bp, Mus musculus
caggttcagctgcagcagtctggggctgagctggtgaagcctggggcctcagtgaagatttcctgcaaagcttctggctacgcattcagtagctactggatgaactgggtgaagcagaggcctggaaagggtcttgagtggattggacagatttatcctggaaatggtgattctaagtacaacggaaagttcaagggcaaggccacactgactgcagacaaatcctccagtacagcctacatgctgctcagcagcctgacctctgaggactctgcggtctatttctgtgcaagatgggaattccggtttgcttactggggccaagggactctggtcactgtctctgca

SEQ ID NO: 123 - DNA, 351 bp, Mus musculus
caggtccaactgcagcagcctggggctgagcttgtgaagcctggggcttcagtgaagatgtcctgcaaggcttctggctacaccttcaccagctactggataacctgggtgaagcagaggcctggacaaggccttgagtggattggagatatttatcctggtagtggtagtactaactacaatgagaagttcaagagcaaggccacactgactgtagacacatcctccagcacagcctacatgcagctcagcagcctgacatctgaggactctgcggtctattactgtgcaagagataactcctatgctatggactactggggtcaaggaacctcagtcaccgtctcctca

SEQ ID NO: 124 - DNA, 354 bp, Mus musculus
cagatccagctgcagcagtctggacctgagctggtgaagcctggggcttcagtgaagatatcctgcaaggcttctggctacaccttcactgactactatataaactgggtgaagcagaggcctgggcagggacttgagtggattggatggatttatcctggaagcggtaatactaagtacaatgagaagttcaagggcaaggccacattgactgtagacacatcctccagcacagcctacatgcagctcagcagcctgacctctgaggactctgcggtctatttctgtgcaagatcttaccgggggtatgctatggactactggggtcaaggaacctcagtcaccgtctcctca

SEQ ID NO: 125 - DNA, 369 bp, Mus musculus
caggtccaactgcagcagcctggggctgagcttgtgaagcctggggcttcagtgaagctgtcctgcaaggcttctggctacaccttcaccagctactggatgcactgggtgaaacagaggcctggacgaggccttgagtggattggaaggattgatcctaatagtggtggtactaagtacaatgagaagttcaagaccaaggccacactgactgtagacaaaccctccagcacagcctacatgcagctcagcagcctgacatctgaggactctgcggtctattattgtgcaagagaggactactatggtaactacttttactggtacttcgatgtctggggcacagggaccacggtcaccgtctcctca

SEQ ID NO: 126 - DNA, 75 bp, Mus musculus
caggtccagctgcagcagtctggacctgagctggtgaagcctggggcttcagtgaagatatcctgcaaggcttct

SEQ ID NO: 127 - DNA, 75 bp, Mus musculus
caggttcagctgcagcagtctggggctgagctggtgaagcctggggcctcagtgaagatttcctgcaaagcttct

SEQ ID NO: 128 - DNA, 75 bp, Mus musculus
caggtccaactgcagcagcctggggctgagcttgtgaagcctggggcttcagtgaagatgtcctgcaaggcttct

SEQ ID NO: 129 - DNA, 75 bp, Mus musculus
cagatccagctgcagcagtctggacctgagctggtgaagcctggggcttcagtgaagatatcctgcaaggcttct

SEQ ID NO: 130 - DNA, 75 bp, Mus musculus
caggtccaactgcagcagcctggggctgagcttgtgaagcctggggcttcagtgaagctgtcctgcaaggcttct

SEQ ID NO: 131 - DNA, 51 bp, Mus musculus
atatattgggtgaagcagaggcctggacagggacttgagtggattggatgg

SEQ ID NO: 132 - DNA, 51 bp, Mus musculus
atgaactgggtgaagcagaggcctggaaagggtcttgagtggattggacag

SEQ ID NO: 133 - DNA, 51 bp, Mus musculus
ataacctgggtgaagcagaggcctggacaaggccttgagtggattggagat

SEQ ID NO: 134 - DNA, 51 bp, Mus musculus
ataaactgggtgaagcagaggcctgggcagggacttgagtggattggatgg

SEQ ID NO: 135 - DNA, 51 bp, Mus musculus
atgcactgggtgaaacagaggcctggacgaggccttgagtggattggaagg

SEQ ID NO: 136 - DNA, 114 bp, Mus musculus
aagtacaatgagaagttcaagggcaaggccacactgacggcagacacatcctccagcactgcctacatgcagctcagcagcctaacatctgaggactctgcggtctattactgt

SEQ ID NO: 137 - DNA, 114 bp, Mus musculus
aagtacaacggaaagttcaagggcaaggccacactgactgcagacaaatcctccagtacagcctacatgctgctcagcagcctgacctctgaggactctgcggtctatttctgt

SEQ ID NO: 138 - DNA, 114 bp, Mus musculus
aactacaatgagaagttcaagagcaaggccacactgactgtagacacatcctccagcacagcctacatgcagctcagcagcctgacatctgaggactctgcggtctattactgt

SEQ ID NO: 139 - DNA, 114 bp, Mus musculus
aagtacaatgagaagttcaagggcaaggccacattgactgtagacacatcctccagcacagcctacatgcagctcagcagcctgacctctgaggactctgcggtctatttctgt

SEQ ID NO: 140 - DNA, 114 bp, Mus musculus
aagtacaatgagaagttcaagaccaaggccacactgactgtagacaaaccctccagcacagcctacatgcagctcagcagcctgacatctgaggactctgcggtctattattgt

SEQ ID NO: 141 - DNA, 33 bp, Mus musculus
tggggtcaaggaacctcagtcaccgtctcctca

SEQ ID NO: 142 - DNA, 33 bp, Mus musculus
tggggccaagggactctggtcactgtctctgca

SEQ ID NO: 143 - DNA, 33 bp, Mus musculus; identical to SEQ ID NO: 141

SEQ ID NO: 144 - DNA, 33 bp, Mus musculus; identical to SEQ ID NO: 141

SEQ ID NO: 145 - DNA, 33 bp, Mus musculus
tggggcacagggaccacggtcaccgtctcctca

SEQ ID NO: 146 - DNA, 24 bp, Mus musculus
ggctacagcttcacaagctactat

SEQ ID NO: 147 - DNA, 24 bp, Mus musculus
ggctacgcattcagtagctactgg

SEQ ID NO: 148 - DNA, 24 bp, Mus musculus
ggctacaccttcaccagctactgg

SEQ ID NO: 149 - DNA, 24 bp, Mus musculus
ggctacaccttcactgactactat

SEQ ID NO: 150 - DNA, 24 bp, Mus musculus
ggctacaccttcaccagctactgg

SEQ ID NO: 151 - DNA, 24 bp, Mus musculus
atttatcctggaagtggtaatact

SEQ ID NO: 152 - DNA, 24 bp, Mus musculus
atttatcctggaaatggtgattct

SEQ ID NO: 153 - DNA, 24 bp, Mus musculus
atttatcctggtagtggtagtact

SEQ ID NO: 154 - DNA, 24 bp, Mus musculus
atttatcctggaagcggtaatact

SEQ ID NO: 155 - DNA, 24 bp, Mus musculus
attgatcctaatagtggtggtact

SEQ ID NO: 156 - DNA, 48 bp, Mus musculus
gcaagatggtcttattacggtagtagctacaactatgctatggactac

SEQ ID NO: 157 - DNA, 27 bp, Mus musculus
gcaagatgggaattccggtttgcttac

SEQ ID NO: 158 - DNA, 30 bp, Mus musculus
gcaagagataactcctatgctatggactac

SEQ ID NO: 159 - DNA, 33 bp, Mus musculus
gcaagatcttaccgggggtatgctatggactac

SEQ ID NO: 160 - DNA, 48 bp, Mus musculus
gcaagagaggactactatggtaactacttttactggtacttcgatgtc

SEQ ID NO: 161 - protein, 712 aa, Mus musculus
MCRIVGAPRTLLPLLAALLQASVEASGEIALCKTGFPEDVYSAVLSQDVHEGQPLLNVKFSNCNGKRKVQYESSEPADFKVDEDGMVYAVRSFPLSSEHAKFLIYAQDKETQEKWQVAVKLSLKPTLPEDSVKESPEIEEIVFPRQATKHNGHLQRQKRDWVIPPINLPENSRGPFPQELVRIRSDRDKNLSLRYSVTGPGADQPPTGIFIINPISGQLSVTKPLDRELIARFHLRAHAVDINGNQVENPIDIVINVIDMNDNRPEFLHQVWNGTVPEGSKPGTYVMTVTAIDADDPNALNGMLRYRILSQAPSTPSPNMFTINNETGDIITVAAGLDREKVQQYTLIIQATDMEGNPTYGLSNTATAVITVTDVNDNPPEFTAMTFYGEVPENRVDVIVANLTVTDKDQPHTPAWNAAYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDV

(19) United States
(12) Patent Application Publication
(10) Pub. No.: US 2021/0024644 A1
(43) Pub. Date: Jan. 28, 2021

(54) N-CADHERIN BINDING MOLECULES AND USES THEREOF

(71) Applicant: Zomedica Pharmaceuticals Corp., Ann Arbor, MI (US)

(72) Inventors: Kevin Gorman, Ann Arbor, MI (US); Maria Dinkelmann, Ann Arbor, MI (US); Allyson Crudgington, Ann Arbor, MI (US); Angy Guerrant, Ann Arbor, MI (US); Casey Wegner, Ann Arbor, MI (US); Stephanie Morley, Ann Arbor, MI (US)

(21) Appl. No.: 16/522,363

(22) Filed: Jul. 25, 2019

(51) Int. Cl. C07K 16/28; G01N 33/574; G01N 33/563

(52) U.S. Cl. CPC C07K 16/2896; G01N 33/563; G01N 33/574

(57) ABSTRACT

Provided herein are antibodies and related molecules that bind to N-cadherin. In particular, provided herein are antibodies and related molecules that binding to canine N-cadherin and uses thereof (e.g., for identifying a variety of different cancer cells).

### FIELD OF THE DISCLOSURE

Provided herein are antibodies and related molecules that bind to N-cadherin. In particular, provided herein are antibodies and related molecules that binding to canine N-cadherin and uses thereof (e.g., for identifying a variety of different cancer cells).

### BACKGROUND OF THE DISCLOSURE

Over the decades, pets moved from the yard to the house to the bed, becoming more and more like another family member every year. Pet owners' willingness to spend money on extending the lives of these precious family members has also increased, but there is a cap to the cost most owners are willing to pay when their pet has been diagnosed with cancer. Veterinary medicine is a cash-based business and requires the ability of the veterinarian, who is the advocate for their patient that cannot speak for itself, to show true value for the medical dollars spent and often maximize on minimal budgets.

Current tools for diagnosing cancer in companion animals are costly because they may require significant capital investment at the point of care (e.g. imaging modalities like ultrasound), surgical biopsy including anesthesia, surgeon time and post-op recovery, or histopathologic examination of the biopsy sample. Moreover, tissue biopsies are plagued by limitations such as invasiveness, lack of procedure repeatability on a patient, and inadequate diagnostic performance. Another problem with the diagnostic process for cancer patients is many animals suffering from cancer are not stable enough for surgical biopsy.

The development of cancer liquid biopsy tests, non-invasive blood testing alternatives to surgical biopsies, is an area of intense focus in human medicine. Cancer liquid biopsy approaches that primarily leverage circulating tumor DNA/RNA (ctDNA and ctRNA) or

CTCs are increasingly being developed for use in diagnostic work-ups and screening in human medicine. However, liquid biopsy offerings have yet to take hold in veterinary medicine. This is likely attributed to a number of factors including cost constraints and a still limited amount of veterinary focused research investigations. A small handful of veterinary companies have developed blood-based cancer tests that rely on approaches such as ELISAs for inflammatory markers and whole blood mRNA signature panels. But these blood tests do not have the necessary diagnostic utility to be used as liquid biopsy tests partially due to the lack of antibodies specific for non-human antigens.

Additional reagents and assays for veterinary applications are needed.

### SUMMARY OF THE DISCLOSURE

Provided herein are antibodies and related molecules that bind to N-cadherin. In particular, provided herein are antibodies and related molecules that binding to canine N-cadherin and uses thereof (e.g., for identifying a variety of different cancer cells).

No circulating tumor cell (CTC) assays have been developed for canine applications. Canine antibodies are typically generated against the human antigen and do not have good enough affinity/performance to be used in detection (e.g., cancer detection) assays. In addition, many existing antibodies to N-cadherin bind to cells other than CTCs (e.g., peripheral blood mononuclear cells (PBMCs)), which leads to inaccuracy in CTC testing.

The antibodies described herein exhibit increased affinity for canine N-cadherin relative to other species (e.g., human) and recognize N-cadherin on circulating tumor cells but not PMBCs. The antibodies find use, for example, in improved methods for detecting cancers in canine subjects.

Accordingly, provided herein is an antibody that specifically binds canine N-cadherin (e.g., the extracellular domain of N-cadherin; amino acids 160-172), wherein the antibody comprises a heavy chain variable region comprising: a CDR1 sequence selected from SEQ ID NOs: 66-70; a CDR2 sequence selected from SEQ ID NOs: 71-75; and a CDR3 sequence selected from SEQ ID NOs: 76-80; and wherein the antibody comprises a light chain variable region comprising: a CDR1 sequence selected from SEQ ID NOs: 26-30; a CDR2 sequence selected from SEQ ID NOs: 31-35; and a CDR3 sequence selected from SEQ ID NOs: 36-40. In some embodiments, the antibody comprises a heavy chain variable region sequence selected from SEQ ID NO: 41-45 and sequences at least 80% (e.g., at least 80%, 85%, 90%, 95%, 98%, or 99%) identical therein and/or wherein the light chain variable region sequence selected from SEQ ID NO: 1-5 and sequences at least 80% (e.g., at least 80%, 85%, 90%, 95%, 98%, or 99%) identical therein. In some embodiments, the antibodies bind to N-cadherin expressed on CTCs but not PBMCs.

Further provided is an isolated polynucleotide encoding a heavy and/or light chain variable region of the antibody described herein, wherein the polynucleotide comprises a nucleic acid comprising: a CDR1 sequence selected from SEQ ID NOs: 146-150; a CDR2 sequence selected from SEQ ID NOs: 151-155; and a CDR3 selected from SEQ ID NOs: 155-159; and wherein the polynucleotide comprises a nucleic acid comprising: a CDR1 sequence selected from SEQ ID NOs: 106-110; a CDR2 sequence selected from SEQ ID NOs: 111-115; and a CDR3 sequence selected from SEQ ID NOs: 116-120. In some embodiments, the polynucleotide comprises a nucleic acid comprising a heavy chain variable region sequence selected from SEQ ID NO: 121-125 and sequences at least 80% (e.g., at least 80%, 85%, 90%, 95%, 98%, or 99%) identical therein and/or wherein the polynucleotide comprises a nucleic acid comprising a light chain variable region sequence selected from SEQ ID NO: 81-85 and sequences at least 80% (e.g., at least 80%, 85%, 90%, 95%, 98%, or 99%) identical therein.

Additional embodiments provide a vector comprising a polynucleotide as described herein, a recombinant host cell comprising a polynucleotide as described herein, or a recombinant host cell comprising a vector as described herein.

Yet other embodiments provide a method of detecting the presence of N-cadherin in a sample, comprising: a) contacting an antibody described herein with the sample; and b) detecting the binding of the antibody to N-cadherin in the sample. In some embodiments, the N-cadherin is expressed on a circulating tumor cell. In some embodiments, the antibody is labeled with a detectable label. In some embodiments, the detecting comprises contacting the antibody with a second labeled antibody that binds to the antibody. In some embodiments, the method is an ELISA assay.

Still other embodiments provide a method of identifying the presence of cancer cells in a biological sample, comprising: a) isolating and capturing circulating tumor cells (CTC) from a biological sample; and b) detecting the presence of N-cadherin on the captured CTCs using an antibody as described herein. In some embodiments, the sample is blood. In some embodiments, the isolating and capturing comprises the use of a microfluidic chip (e.g., wherein detecting is performed on the captured CTCs in the microfluidic chip). In some embodiments, method further comprises detecting the presence of captured white blood cells (WBCs) (e.g., by detecting the presence of a CD45 polypeptide on captured WBC).

The present disclosure is not limited to detection of a particular cancer. Examples include, but not limited to, hemangiosarcoma, osteosarcoma, mammary cancers, mixed cancers, or carcinomas. In some embodiments, the assay identifies the presence of any one of the cancers (e.g., but does not distinguish between the different cancers). In some embodiments, the sample is obtained from a non-human subject (e.g., a canine subject).

In certain embodiments, provided herein is a kit or system, comprising: an antibody as described herein. In some embodiments, the kit or system further comprises an antibody that specifically binds to CD45.

In other embodiments, the present disclosure provides a kit or system as described herein for use in detecting the presence of cancer in a biological sample or the use of a kit or system as described herein for detecting the presence of cancer in a biological sample.

In still other embodiments, the present disclosure provides a kit or system as described herein for use in detecting the presence of N-cadherin in a sample or the use of a kit or system as described herein for detecting the presence of N-cadherin in a sample.

Certain embodiments provide the use of an antibody as described herein for detecting the presence of cancer in a biological sample or an antibody as described herein for use in detecting the presence of N-cadherin in a sample.

Additional embodiments are described herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows titer testing for immunized-mouse sera. (A/B) Reactivity with canine osteosarcoma cell lines, and (C) canine PBMCs.

FIG. 2 shows results of a secondary flow cytometry screen with fusion clones.

FIG. 3 shows sequences of exemplary antibodies described herein.

FIG. 4 shows the sequence of n-cadherin (SEQ ID NO:161).

### DEFINITIONS

To facilitate an understanding of the present disclosure, a number of terms and phrases are defined below:

As used herein, the terms “detect”, “detecting”, or “detection” may describe either the general act of discovering or discerning or the specific observation of a composition.

The expression “amino acid position corresponding to” a position in a reference sequence and similar expression is intended to identify the amino acid residue that in the primary or spatial structure corresponds to the particular position in the reference sequence.

This can be done by aligning a given sequence with the reference sequence and identifying the amino acid residue that aligns with the particular position in the reference sequence.

The term “sample” as used herein is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present disclosure.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries.

As used herein “immunoglobulin” refers to any class of structurally related proteins in the serum and the cells of the immune system that function as antibodies. In some embodiments, an immunoglobulin is the distinct antibody molecule secreted by a clonal line of B cells.

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc. A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “anti-n-cad antibody” or “n-cad antibody” refers to an antibody which specifically recognizes an antigen and/or epitope presented by N-cadherin.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods; See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety. As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

As used herein, the term “chimeric antibody” refers to an antibody made up of components from at least two different sources. In certain embodiments, a chimeric antibody comprises a portion of an antibody derived from a first species fused to another molecule, e.g., a portion of an antibody derived from a second species. In certain such embodiments, a chimeric antibody comprises a portion of an antibody derived from a non-human animal fused to a portion of an antibody derived from a human. In certain such embodiments, a chimeric antibody comprises all or a portion of a variable region of an antibody derived from a non-human animal fused to a constant region of an antibody derived from a human.

As used herein, the term “natural antibody” refers to an antibody in which the heavy and light chains of the antibody have been made and paired by the immune system of a multicellular organism. For example, the antibodies produced by the antibody-producing cells isolated from a first animal immunized with an antigen are natural antibodies. Natural antibodies contain naturally-paired heavy and light chains. The term “natural human antibody” refers to an antibody in which the heavy and light chains of the antibody have been made and paired by the immune system of a human subject.

Native human light chains are typically classified as kappa and lambda light chains. Native human heavy chains are typically classified as mu, delta, gamma, alpha, or epsilon, and define the antibody's isotype as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids. See, e.g., Fundamental Immunology (1989) Ch. 7 (Paul, W., ed., 2nd ed. Raven Press, N.Y.); herein incorporated by reference in its entirety.

The term “antigen-binding site” refers to a portion of an antibody capable of specifically binding an antigen. In certain embodiments, an antigen-binding site is provided by one or more antibody variable regions.

The term “epitope” refers to any polypeptide determinant capable of specifically binding to an immunoglobulin or a T-cell or B-cell receptor. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three dimensional structural characteristics (e.g., a “conformational” epitope) and/or specific charge characteristics.

As used herein, the term “multivalent”, particularly when used in describing an agent that is an antibody, antibody fragment, or other binding agent, refers to the presence of two or more (e.g., 2, 3, 4, 5, 6, 7, 8, 9, 10, or more) antigen binding sites on the agent. As used herein, the term “multispecific”, particularly when used in describing an agent that is an antibody, antibody fragment, or other binding agent, refers to the capacity to of the agent to bind two or more (e.g., 2, 3, 4, 5, 6, 7, 8, 9, 10, or more) targets (e.g., unrelated targets). For example, a bispecific antibody recognizes and binds to two different antigens. An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties. Naturally occurring residues may be divided into classes based on common side chain properties, for example: hydrophobic: norleucine, Met, Ala, Val, Leu, and Ile; neutral hydrophilic: Cys, Ser, Thr, Asn, and Gln; acidic: Asp and Glu; basic: His, Lys, and Arg; residues that influence chain orientation: Gly and Pro; and aromatic: Trp, Tyr, and Phe. Non-conservative substitutions may involve the exchange of a member of one of these classes for a member from another class; whereas conservative substitutions may involve the exchange of a member of one of these classes for another member of that same class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families (see above). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “selectively” (e.g., as in “selectively targets,” “selectively binds,” etc.) refers to the preferential association of an agent (e.g., antibody or antibody fragment) for a particular entity (e.g., antigen, antigen presenting cell, etc.). For example, an agent selectively targets a particular cell population if it preferentially associates (e.g., binds an epitope or set of epitopes presented thereon) with that cell population over another cell population (e.g., all other cell populations present in a sample). The preferential association may be by a factor of at least 2, 4, 6, 8, 10, 20, 50, 100, 10³, 10⁴, 10⁵, 10⁶, or more, or ranges there between. An agent that X-fold selectively targets a particular cell populations, associates with that cell population by at least X-fold more than other cell populations present.

### DETAILED DESCRIPTION OF THE DISCLOSURE

Provided herein are antibodies to N-cadherin. In particular, provided herein are antibodies to canine N-cadherin and uses thereof (e.g., for identifying a variety of different cancer cells).

In experiments conducted during the course of development of embodiments of the present disclosure, a number of commercially-available human antibodies that have been validated for use in canines were tested in a liquid biopsy assay for cancer. However, none of the commercially-available antibodies were suitable for one of two reasons: either the antibody did not adequately stain cancer cells, or it was promiscuous and stained other circulating cells, thereby creating false positives.

Accordingly, provided herein are monoclonal antibodies and related molecules (e.g., mouse monoclonal antibodies and fragments thereof) with the following characteristics: improved affinity for the canine N-cadherin compared to commercially-available products, such that they stain CTCs; and enhanced specificity for the canine antigen over other circulating cells.

### I. Antibodies

As described above, embodiments of the present disclosure provide antibodies that specifically bind canine N-cadherin.

In some embodiments, the immunoglobulin molecule is composed of two identical heavy and two identical light polypeptide chains, held together by interchain disulfide bonds. Each individual light and heavy chain folds into regions of about 110 amino acids, assuming a conserved three-dimensional conformation. The light chain comprises one variable region (termed VL) and one constant region (CL), while the heavy chain comprises one variable region (VH) and three constant regions (CH1, CH2 and CH3). Pairs of regions associate to form discrete structures. In particular, the light and heavy chain variable regions, VL and VH, associate to form an “FV” area that contains the antigen-binding site.

The variable regions of both heavy and light chains show considerable variability in structure and amino acid composition from one antibody molecule to another, whereas the constant regions show little variability. Each antibody recognizes and binds an antigen through the binding site defined by the association of the heavy and light chain, variable regions into an FV area. The light-chain variable region VL and the heavy-chain variable region VH of a particular antibody molecule have specific amino acid sequences that allow the antigen-binding site to assume a conformation that binds to the antigen epitope recognized by that particular antibody.

Within the variable regions are found regions in which the amino acid sequence is extremely variable from one antibody to another. Three of these so-called “hypervariable” regions or “complementarity-determining regions” (CDR's) are found in each of the light and heavy chains. The three CDRs from a light chain and the three CDRs from a corresponding heavy chain form the antigen-binding site.

The amino acid sequences of many immunoglobulin heavy and light chains have been determined and reveal two important features of antibody molecules. First, each chain consists of a series of similar, although not identical, sequences, each about 110 amino acids long. Each of these repeats corresponds to a discrete, compactly folded region of protein structure known as a protein domain. The light chain is made up of two such immunoglobulin domains, whereas the heavy chain of the IgG antibody contains four.

The second important feature revealed by comparisons of amino acid sequences is that the amino-terminal sequences of both the heavy and light chains vary greatly between different antibodies. The variability in sequence is limited to approximately the first 110 amino acids, corresponding to the first domain, whereas the remaining domains are constant between immunoglobulin chains of the same isotype. The amino-terminal variable or V domains of the heavy and light chains (V_{H} and V_{L}, respectively) together make up the V region of the antibody and confer on it the ability to bind specific antigen, while the constant domains (C domains) of the heavy and light chains (C_{H} and C_{L}, respectively) make up the C region. The multiple heavy-chain C domains are numbered from the amino-terminal end to the carboxy terminus, for example C_{H}1, C_{H}2, and so on.

The protein domains described above associate to form larger globular domains. Thus, when fully folded and assembled, an antibody molecule comprises three relatively equal-sized globular portions joined by a flexible stretch of polypeptide chain known as the hinge region. Each arm of this Y-shaped structure is formed by the association of a light chain with the amino-terminal half of a heavy chain, whereas the trunk of the Y is formed by the pairing of the carboxy-terminal halves of the two heavy chains. The association of the heavy and light chains is such that the V_{H} and V_{L} domains are paired, as are the C_{H}1 and C_{L} domains. The C_{H}3 domains pair with each other but the C_{H}2 domains do not interact; carbohydrate side chains attached to the C_{H}2 domains lie between the two heavy chains. The two antigen-binding sites are formed by the paired V_{H} and V_{L} domains at the ends of the two arms of the Y.

Proteolytic enzymes (proteases) that cleave polypeptide sequences have been used to dissect the structure of antibody molecules and to determine which parts of the molecule are responsible for its various functions. Limited digestion with the protease papain cleaves antibody molecules into three fragments. Two fragments are identical and contain the antigen-binding activity. These are termed the Fab fragments, for Fragment antigen binding. The Fab fragments correspond to the two identical arms of the antibody molecule, which contain the complete light chains paired with the V_{H} and C_{H}1 domains of the heavy chains. The other fragment contains no antigen-binding activity but was originally observed to crystallize readily, and for this reason was named the Fc fragment, for Fragment crystallizable. This fragment corresponds to the paired C_{H}2 and C_{H}3 domains and is the part of the antibody molecule that interacts with effector molecules and cells. The functional differences between heavy-chain isotypes lie mainly in the Fc fragment. The hinge region that links the Fc and Fab portions of the antibody molecule is in reality a flexible tether, allowing independent movement of the two Fab arms, rather than a rigid hinge.

In some embodiments, provided herein is an antibody that specifically binds canine N-cadherin (e.g., the extracellular domain of N-cadherin; amino acids 160-172). In some embodiments, the antibody binds to N-cadherin expressed on CTCs but not PBMCs.

In some embodiments, the heavy chain of the antibody comprises a CDR1 sequence selected from SEQ ID NOs: 66-70; a CDR2 sequence selected from SEQ ID NOs: 71-75; and a CDR3 sequence selected from SEQ ID NOs: 76-80; and the light chain of the antibody comprises a CDR1 sequence selected from SEQ ID NOs: 26-30; a CDR2 sequence selected from SEQ ID NOs: 31-35; and a CDR3 sequence selected from SEQ ID NOs: 36-40.

The antibodies are not limited to particular heavy and light chain variable region sequences. In some embodiments, the antibody comprises, consists essentially of, or consists of a heavy chain variable region sequence selected from SEQ ID NO: 41-45 and sequences at least 80% (e.g., at least 80%, 85%, 905, 95%, 98%, or 99%) identical therein and a light chain variable region sequence selected from SEQ ID NO: 1-5 and sequences at least 80% (e.g., at least 80%, 85%, 905, 95%, 98%, or 99%) identical therein.

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

In some embodiments, the antibody is a chimeric antibody (e.g., comprising a variable region or CDR sequences described herein and a different constant region). In some embodiments, chimeras comprise constant region sequences from a different species or isotype as described herein. In some embodiments, the antibody is a fragment (e.g., a fragment that retains binding to canine N-cadherin).

Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

Further provided is an isolated polynucleotide encoding a heavy and/or light chain variable region of the antibody described herein. Such nucleic acids find use in production and/or screening of antibodies. In some embodiments, the polynucleotide comprises a nucleic acid comprising: a CDR1 sequence selected from SEQ ID NOs: 146-150; a CDR2 sequence selected from SEQ ID NOs: 151-155; and a CDR3 selected from SEQ ID NOs: 155-159; and wherein the polynucleotide comprises a nucleic acid comprising: a CDR1 sequence selected from SEQ ID NOs: 106-110; a CDR2 sequence selected from SEQ ID NOs: 111-115; and a CDR3 sequence selected from SEQ ID NOs: 116-120. In some embodiments, the polynucleotide comprises a nucleic acid comprising a heavy chain variable region sequence selected from SEQ ID NO: 121-125 and sequences at least 80% (e.g., at least 80%, 85%, 90%, 95%, 98%, or 99%) identical therein and/or wherein the polynucleotide comprises a nucleic acid comprising a light chain variable region sequence selected from SEQ ID NO: 81-85 and sequences at least 80% (e.g., at least 80%, 85%, 90%, 95%, 98%, or 99%) identical therein.

Addition embodiments provide a vector comprising a polynucleotide as described herein, a recombinant host cell comprising a polynucleotide as described herein, or a recombinant host cell comprising a vector as described herein.

The disclosure also features methods for producing any of the antibodies or antigen-binding fragments thereof described herein. In some embodiments, methods for preparing an antibody described herein can include immunizing a subject (e.g., a non-human mammal) with an appropriate immunogen. For example, to generate an antibody that binds to N-cadherin, one can immunize a suitable subject (e.g., a non-human mammal such as a rat, a mouse, a gerbil, a hamster, a dog, a cat, a pig, a goat, a horse, or a non-human primate) with a full-length or fragment of an N-cadherin polypeptide.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a N-cadherin polypeptide as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybridoma cells are then screened for secretion of the desired antibodies, e.g., an antibody that binds to canine N-cadherin.

In some embodiments, an N-cadherin antibody is identified from a non-immune biased library as described in, e.g., U.S. Pat. No. 6,300,064 (to Knappik et al.; Morphosys AG) and Schoonbroodt et al. (2005) Nucleic Acids Res 33(9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

In phage display methods, functional antibody domains are displayed on the surface of phage particles which carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen binding domains are expressed as a recombinantly-fused protein to any of the phage coat proteins pIII, pVIII, or pIX. See, e.g., Shi et al. (2010) JMB 397:385-396. Examples of phage display methods that can be used to make the immunoglobulins, or fragments thereof, described herein include those disclosed in Brinkman et al. (1995) J Immunol Methods 182:41-50; Ames et al. (1995) J Immunol Methods 184:177-186; Kettleborough et al. (1994) Eur J Immunol 24:952-958; Persic et al. (1997) Gene 187:9-18; Burton et al. (1994) Advances in Immunology 57:191-280; and PCT publication nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

In some embodiments, the phage display antibody libraries can be generated using mRNA collected from B cells from the immunized mammals. For example, a splenic cell sample comprising B cells can be isolated from mice immunized with a N-cadherin polypeptide as described above. mRNA can be isolated from the cells and converted to cDNA using standard molecular biology techniques. See, e.g., Sambrook et al. (1989) “Molecular Cloning: A Laboratory Manual, 2.sup.nd Edition,” Cold Spring Harbor Laboratory Press, Cold Spring Harbor, N.Y.; Harlow and Lane (1988), supra; Benny K. C. Lo (2004), supra; and Borrebaek (1995), supra. The cDNA coding for the variable regions of the heavy chain and light chain polypeptides of immunoglobulins are used to construct the phage display library. Methods for generating such a library are described in, e.g., Merz et al. (1995) J Neurosci Methods 62(1-2):213-9; Di Niro et al. (2005) Biochem J 388(Pt 3):889-894; and Engberg et al. (1995) Methods Mol Biol 51:355-376.

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al., (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen (e.g., canine N-cadherin) using any immunological or biochemical based method. For example, specific binding of an antibody to canine N-cadherin, may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays.

In embodiments where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody. Alternatively, fragments of chemically synthesized nucleic acids, together capable of encoding an antibody, can be joined together using DNA assembly techniques.

The antibodies or antigen-binding fragments thereof described herein can be produced using a variety of techniques in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof are expressed in, and purified from, transgenic animals (e.g., transgenic mammals). For example, an antibody is produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3.sup.rd edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

Further embodiments provide compositions, kits, and systems. Compositions for use in the diagnostic methods of the present disclosure include, but are not limited to, antibodies (e.g., N-cadherin antibodies described herein), antibodies for identifying WBCs, detection reagents, and sample preparation reagents.

Any of these compositions, alone or in combination with other compositions of the present disclosure, may be provided in the form of a kit. In some embodiments, antibodies and reagents are provided in one or more containers. Kits may further comprise appropriate controls and/or detection reagents. For example, in some embodiments, kits comprise one or more of a multiwell plate, lateral flow strips, beads, analysis software, and the like.

II. Uses The antibodies described herein find use in a variety of applications. In some embodiments, the antibodies are used to detect the present of canine N-cadherin in a sample (e.g., a biological or clinical sample, although other sample types are specifically contemplated). In some embodiments, detection of N-cadherin comprises an immunoassay (e.g., those described below).

Illustrative non-limiting examples of immunoassays include, but are not limited to:

immunoprecipitation; Western blot; ELISA; immunohistochemistry; immunocytochemistry; flow cytometry; and, immuno-PCR. Polyclonal or monoclonal antibodies detectably labeled using various techniques (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, immunomagnetic detection is utilized. In some embodiments, detection is automated. Exemplary immunomagnetic detection methods include, but are not limited to, those commercially available from Veridex (Raritan, N.J.).

In some embodiments, the antibodies described herein find use in diagnosing or characterizing cancer.

In some embodiments, the present disclosure utilizes N-cadherin cancer markers that identify a wide variety of cancers. For example, in some embodiments, the assays described herein detect the presence of one or more of hemangiosarcoma, osteosarcoma, mammary cancers, mixed cancer tumors and carcinomas.

In some embodiments, the presence of cancer is determined by detecting the presence of N-cadherin on a circulating tumor cell (CTC).

In some embodiments, the CTC assay described in U.S. patent application Ser. No. 16/409,408, herein incorporated by reference in its entirety, is utilized. Briefly, in some embodiments, the assays of the present disclosure comprise a first step of isolating CTCs from a biological sample (e.g., blood or other biological sample), followed by identification of tumor markers (e.g., N-cadherin) associated with the CTCs.

The present disclosure is not limited to particular methods for capture and analysis of CTCs. In some embodiments, methods of the present disclosure utilize commercially available systems for isolation and/or characterization of CTCs. Examples include, but are not limited to, the CellSearch™ system, (Immunicon Corporation, Huntingdon Valley, Pa.) (Allard et al., Clin Cancer Res 2004;10(20):6897-904; Cristofanilli et al., N Engl J Med 2004;351(8):781-91; each of which is herein incorporated by reference in its entirety) and Celsee (Plymouth, Mich.) (Gogoi et al., 2016 and U.S. Pat. No 9,404,864); each of which is herein incorporated by reference in its entirety.

In some embodiments, commercially available systems from Seraph Biosciences (Detroit, Mich.) or Qorvo (Greensboro, N.C.) are utilized.

In some embodiments, assays comprise the steps of preparing a sample, isolating the sample, and detecting the presence of cancer markers associated (e.g., on the surface or inside) the CTCs. In some embodiments, the presence of captured white blood cells is determined using antibodies to CD45. Such white blood cells are then excluded from further analysis. In some embodiments, automated sample preparation, capture, and analysis is performed. In some embodiments, software is used to identify captured CTCs that express the cancer markers described herein.

In some embodiments, the assays find use in diagnostic methods for identifying cancer in a sample from a subject. In some embodiments, the subject is a non-human animal. In some embodiments, the non-human animal is a companion animal (e.g., dog, cat, etc.). The present disclosure is illustrated with canine samples. However, it is specifically contemplated that the described methods can be used to detect cancer cells in samples from other companion or non-companion animals.

In some embodiments, a computer-based analysis program is used to translate the raw data generated by the detection assay (e.g., the presence, absence, or amount of cancer marker) into data of predictive value for a clinician (e.g., presence of cancer). The clinician can access the predictive data using any suitable means. Thus, in some preferred embodiments, the present disclosure provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present disclosure contemplates any method capable of receiving, processing, and transmitting the information to and from laboratories conducting the assays, information provides, medical personal, and subjects. For example, in some embodiments of the present disclosure, a sample (e.g., blood sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained (e.g., by a veterinary nurse) and sent to the profiling center, or subjects or pet owners may collect the sample themselves (e.g., a urine sample) and directly send it to a profiling center. Once received by the profiling service, the sample is processed and a profile is produced (i.e., cancer marker data), specific for the diagnostic or prognostic information desired for the subject.

The profile data is then prepared in a format suitable for interpretation by a treating clinician. For example, rather than providing raw data, the prepared format may represent a diagnosis (e.g., presence of cancer) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some exemplary embodiments, the sample (e.g., blood sample) is first obtained at the point of care (e.g., by a veterinary nurse), placed in a suitable container (e.g., vacuum blood tube), labeled with a unique identifier, and then sent to a testing lab (e.g., reference lab) by any suitable method. In some embodiments, the testing lab performs the analysis (e.g., using an automated system described herein) and provided results to the point of care provider in any suitable format (e.g., using an electronic portal). In some embodiments, depending on the analysis method, further sample preparation is performed at the point of care or testing laboratory (centrifugation).

In some exemplary embodiments, the sample (e.g., stool or urine sample) is first obtained at the point of care (e.g., by a veterinary nurse), placed in a suitable container (e.g., cuvette), labeled with a unique identifier, and then sent to a testing lab (e.g., reference lab) by any suitable method. In some embodiments, the testing lab performs the analysis (e.g., using an automated system suitable for analysis of urine or stool samples) and provided results to the point of care provider in any suitable format (e.g., using an electronic portal).

In some embodiments, all of the analysis is performed at the point of care (e.g., using an automated analysis system).

In some embodiments, the subject or pet owner is able to directly access the data using the electronic communication system. The subject or pet owner may chose further intervention or counseling based on the results. In some embodiments, the data is used for research use. For example, the data may be used to further optimize the inclusion or elimination of markers as useful indicators of a particular condition or stage of disease.

### Experimental

The following examples are provided to demonstrate and further illustrate certain embodiments of the present disclosure and are not to be construed as limiting the scope thereof.

### EXAMPLE 1

### Materials and Methods

### In Vivo Immunization

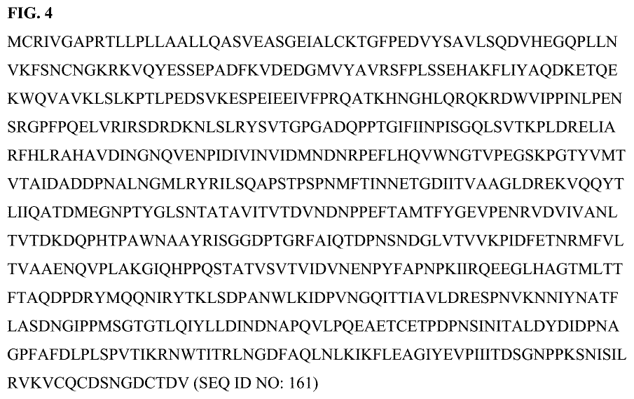

The pcDNA3.1(+) vector was modified to code for the canine N-cadherin full length sequence (GenScript). The amino acid sequence (SEQ ID NO:161) used in the vector is shown in FIG. 4. The amino acid sequence was codon-optimized for expression in mice. The natural signal sequence (AA 1-159) is used but the absence of a membrane anchoring domain and allows the protein to be released from the cell in a soluble form. These sequences are cleaved prior to the protein being secreted. Thus, antibodies were generated against the extracellular domain (AA 160-172 of SEQ ID NO:161).

Eight mice were injected with 50 μg of vector DNA each. After injection, endogenous mechanisms performed transcription and translation of the protein product (antigen) encoded in the vector. Mice were boosted every week thereafter for 6 weeks with an additional 50 μg of vector DNA.

### Titer Test

Wells were seeded with 100,000 cells each from OSCA-8 and D-17 cell lines. The cells were then resuspended in 50 μL of titrated serum (starting at 1:100, 3× dilutions for 11 points) and incubated for 1 hour on ice. Then, 50 μL of secondary antibody was added, and the mixture was allowed to incubate for 30 minutes on ice. The mixtures were then fixed with 1% PFA, and subsequently analyzed via flow cytometry (at least 5000 events). Normal mouse serum was used as a negative control.

### Fusion Procedure

### Screening Fusion Clones by Flow Cytometry

Wells were seeded with 100,000 cells from each of the following cell types: D17, OSCA-8, EFB, and canine PBMCs. Each cell type was then resuspended in 100 μL of culture media from a hybridoma (which contains secreted monoclonal antibody), and incubated for 1 hour on ice. Then, 50 μL of secondary antibody was added, and the mixture was allowed to incubate for 30 minutes on ice. The mixtures were then fixed with 1% PFA, and subsequently analyzed via flow cytometry (at least 5000 events). Normal mouse serum was used as a negative control.

### Results

### Examination of Mouse Sera After Immunization

An in vivo immunization scheme was used whereby a specialized expression vector pcDNA3.1(+) was modified to contain a codon-optimized nucleic acid sequence corresponding to the canine N-cadherin protein sequence.

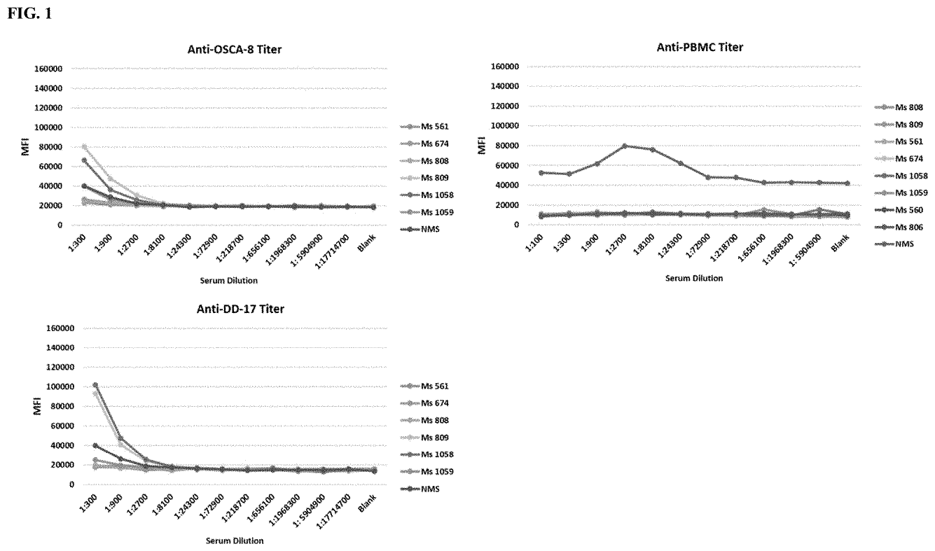

Seven weeks after immunization, sera from the immunized mice was analyzed to determine if a given mouse had generated antibodies against the antigen. Sera was used in flow cytometry to determine if antibodies therein could bind to canine cancer cells, and, importantly, not react with PBMCs in flow cytometry (FIG. 1). Two mice were seropositive for cancer cells and seronegative for binding to PBMCs (Ms809 and Ms1058), and were therefore selected for fusion.

### Post-Fusion Screening

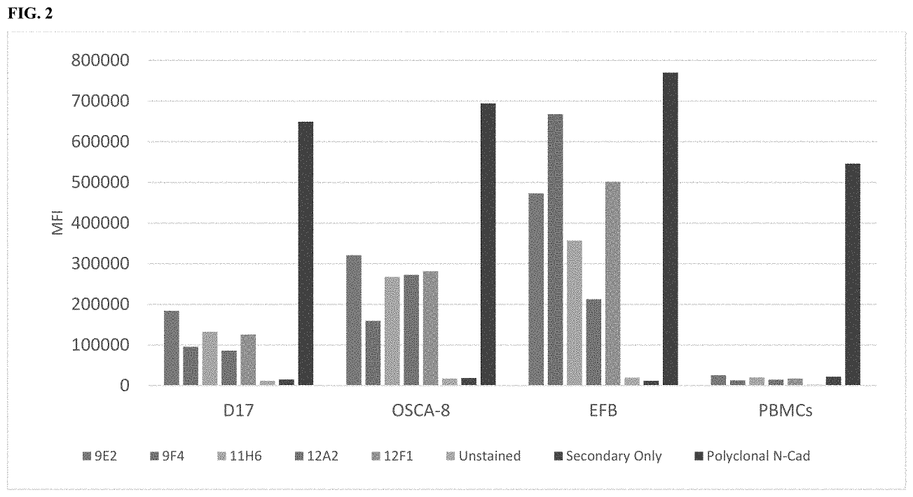

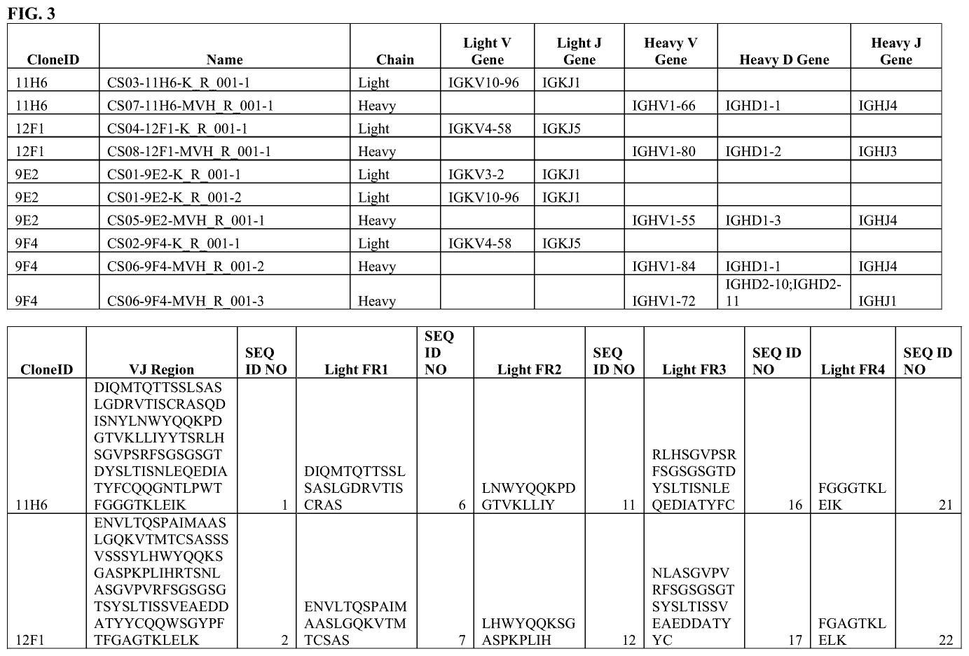

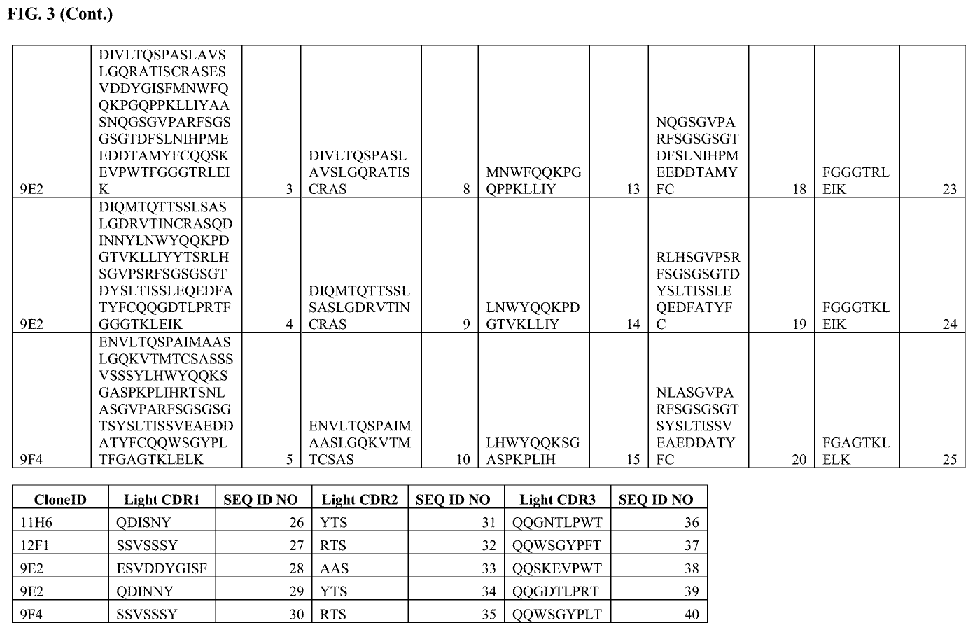

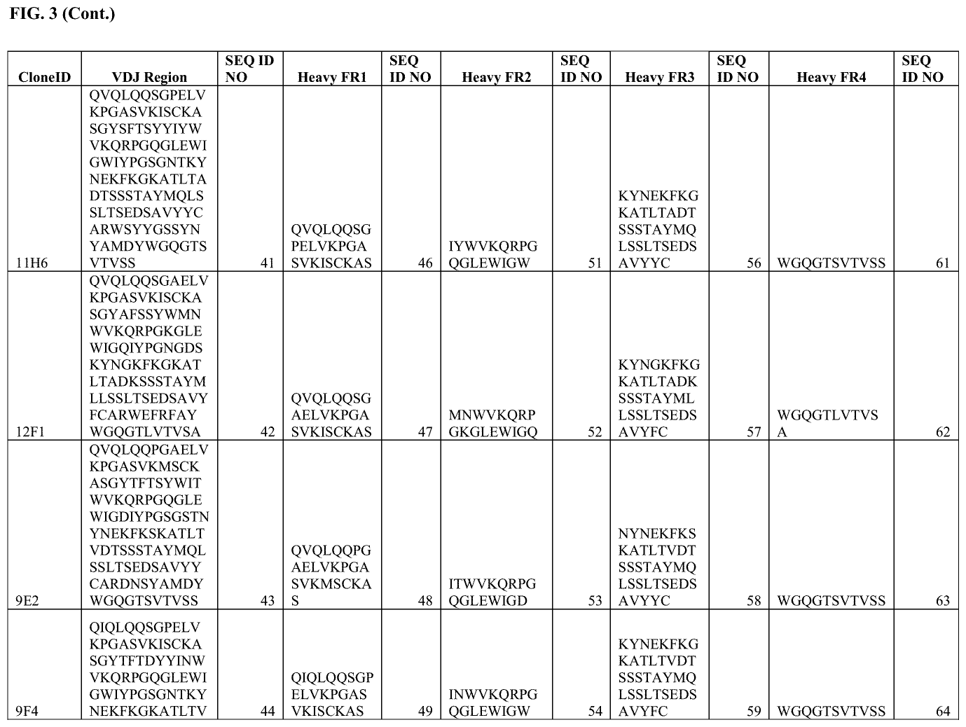

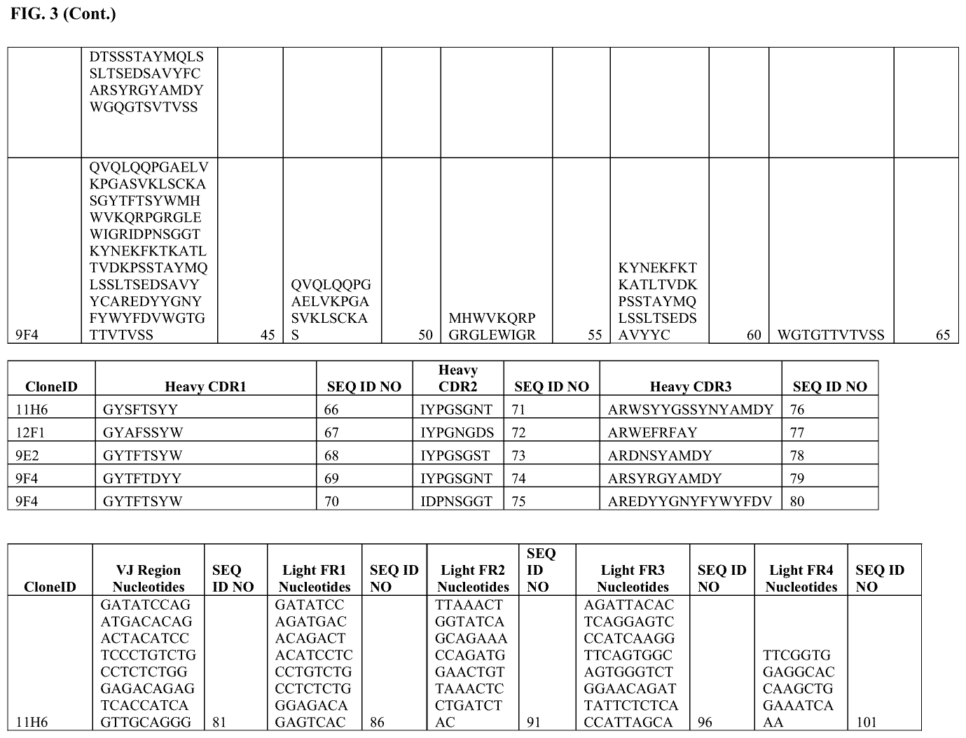

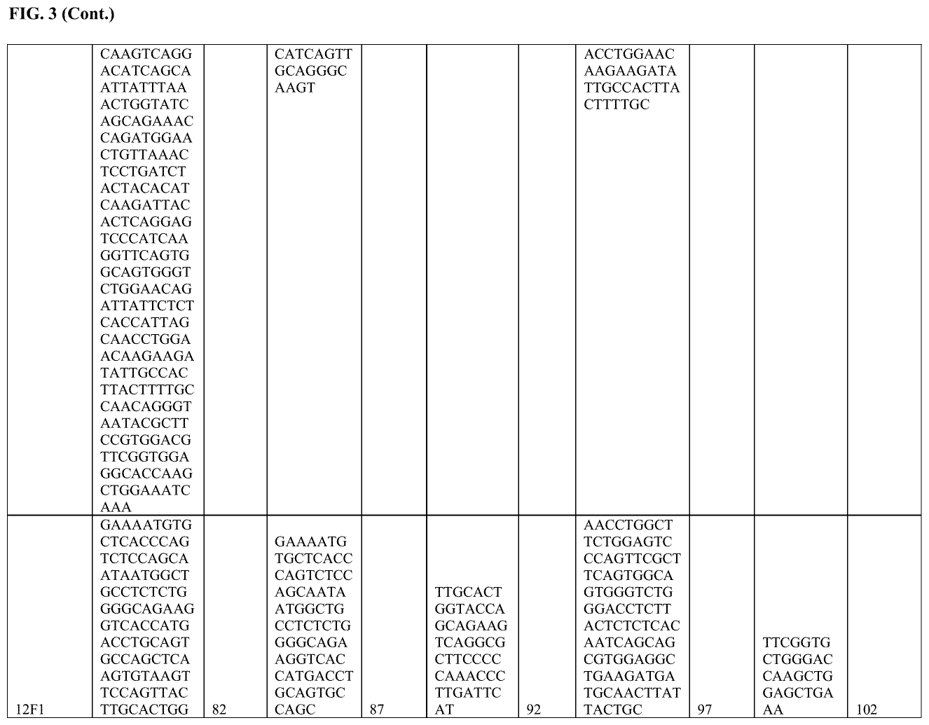

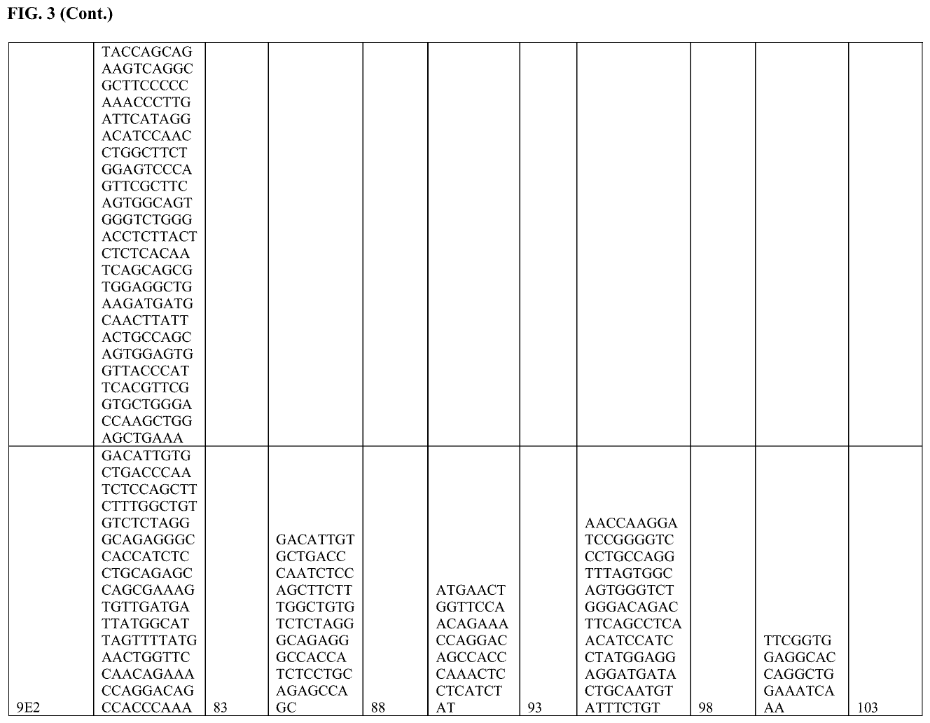

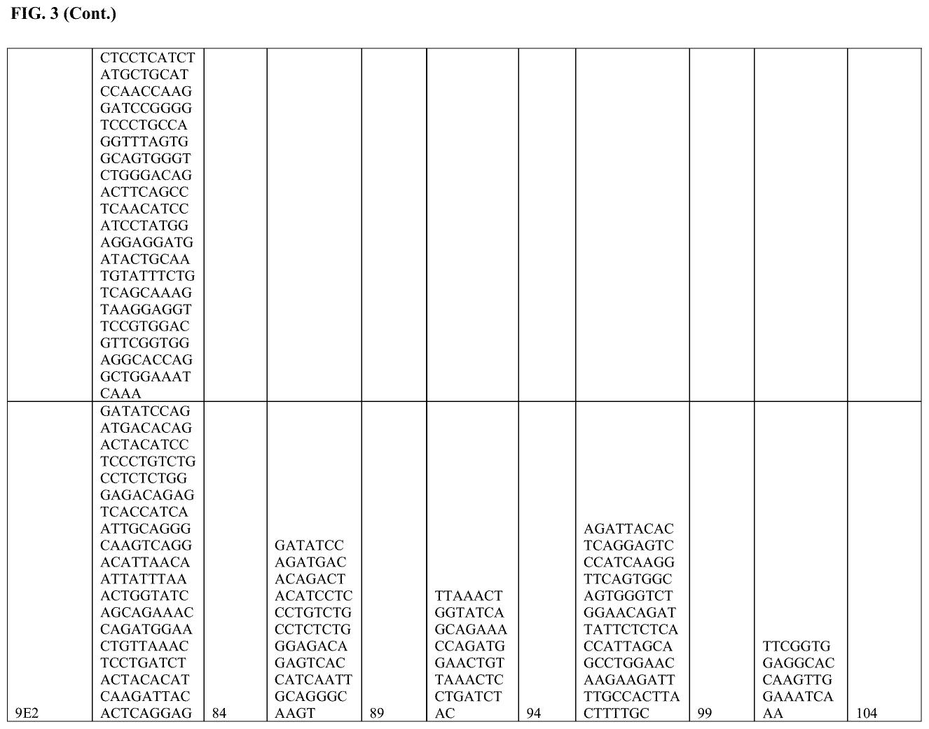

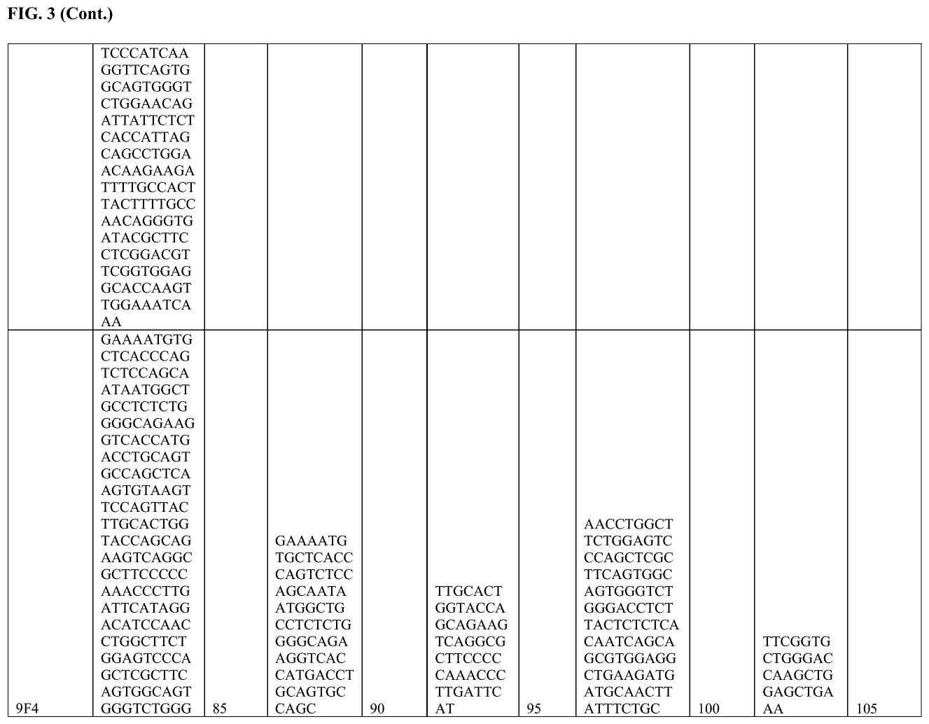

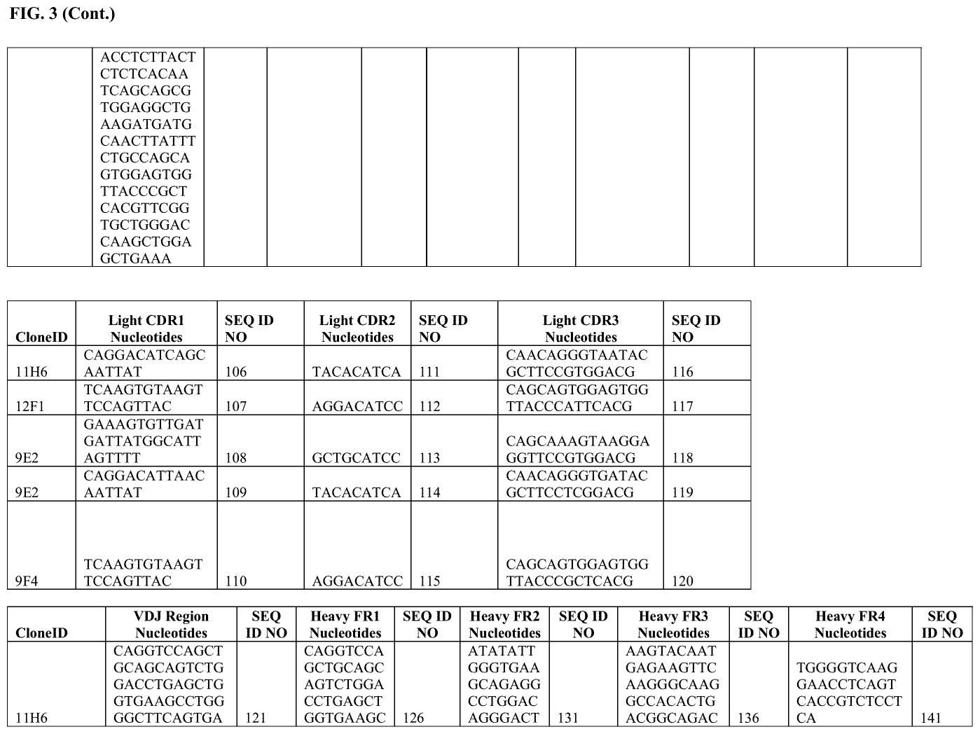

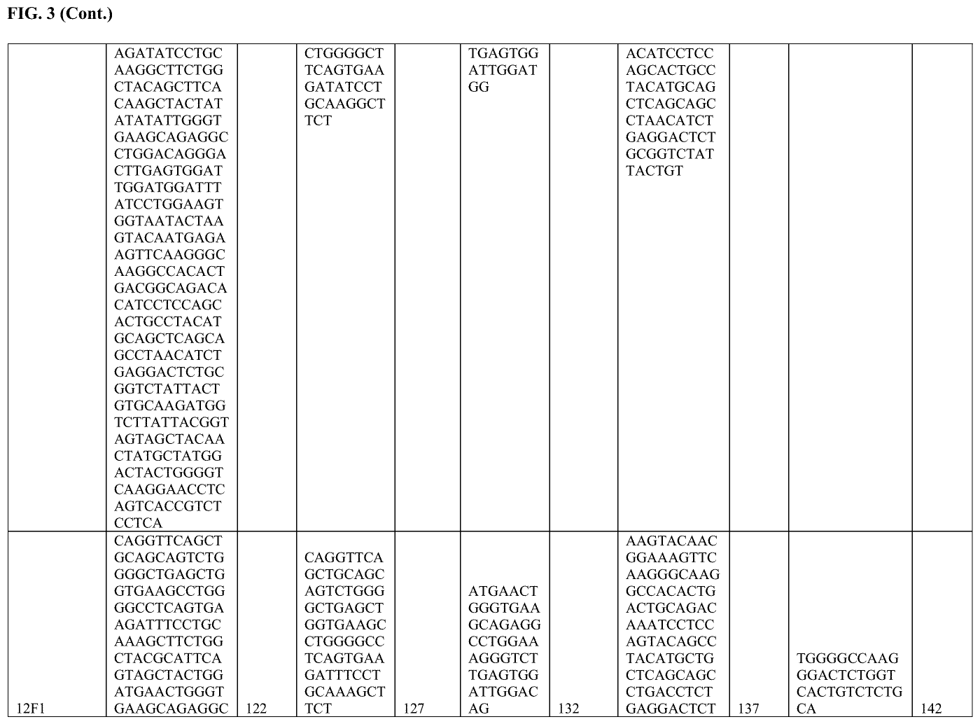

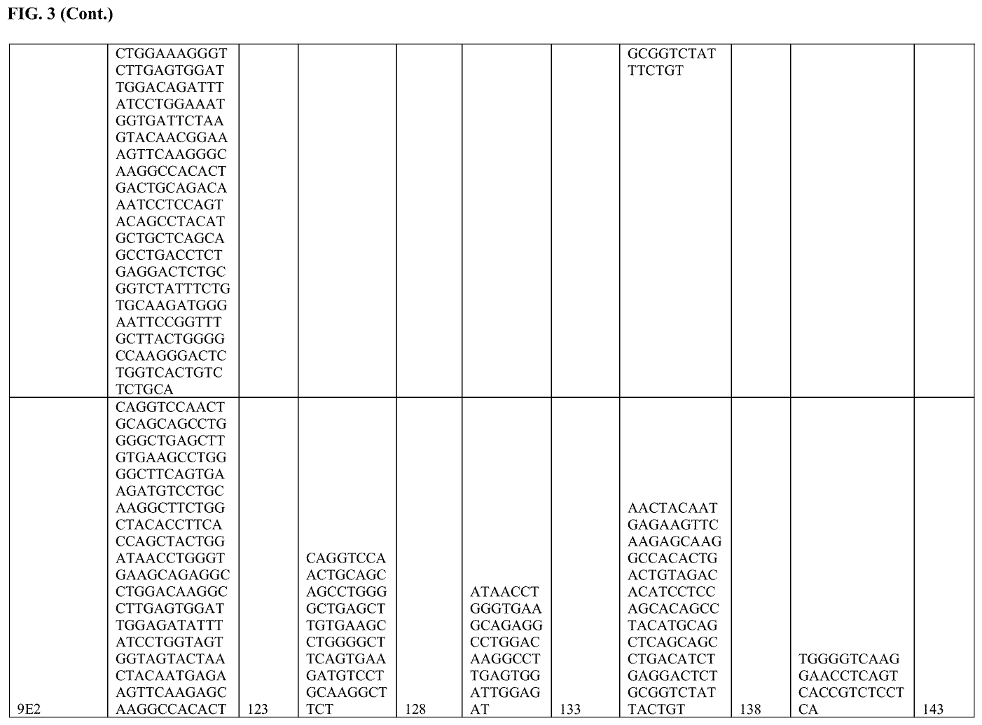

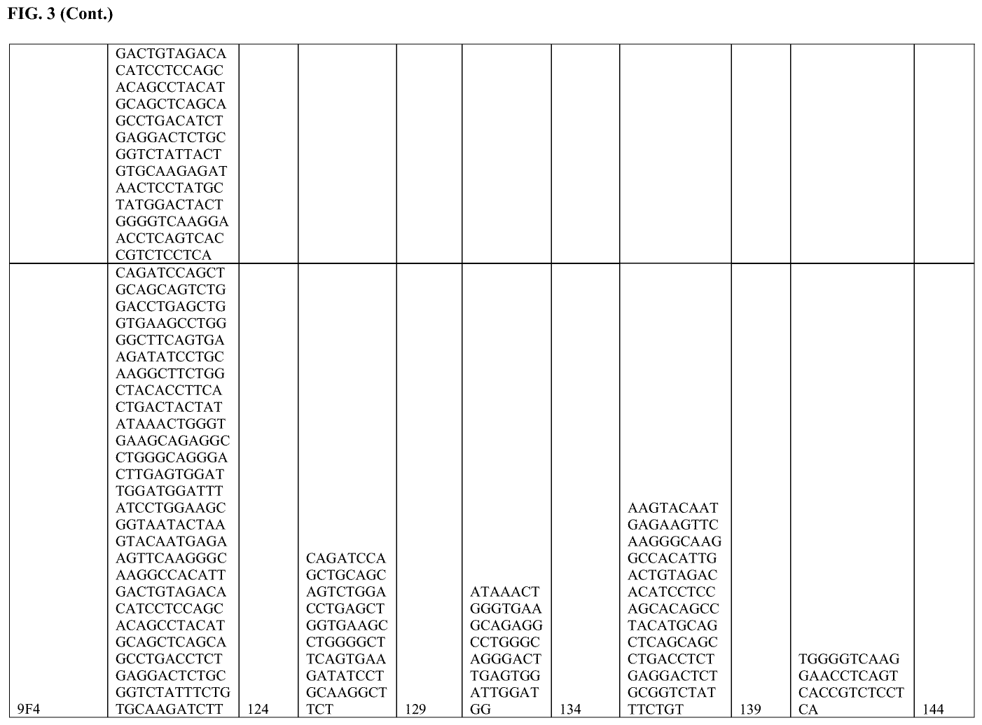

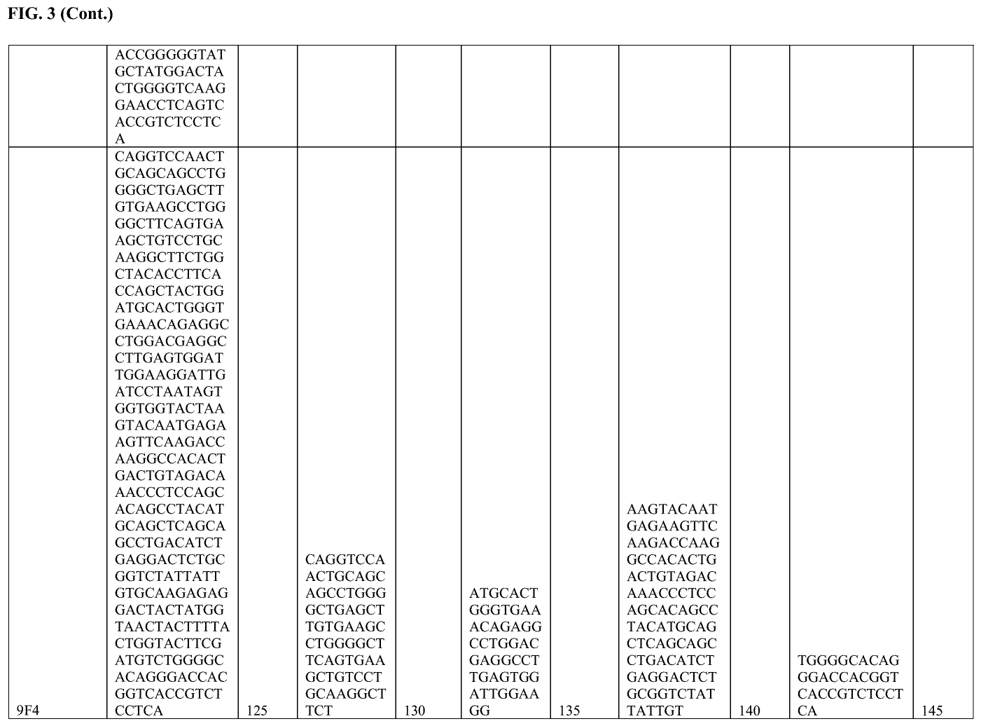

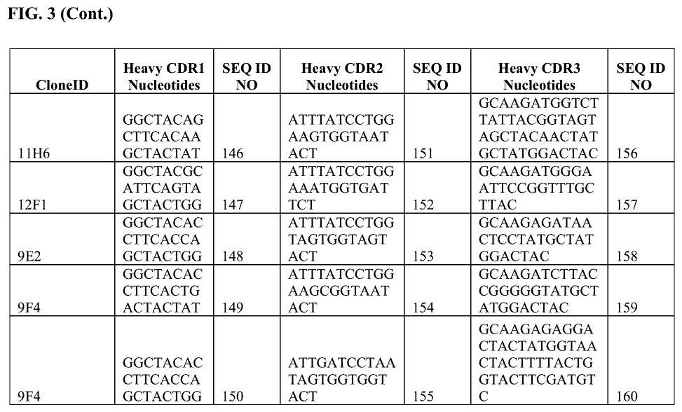

After fusion, a primary screen was performed where supernatants from fusions were used in flow cytometry to determine if they could label a 1:1 mixture of both canine osteosarcoma cell lines (D17 and OSCAR). Of the 2,208 clones screened, 26 positive hits were identified. A secondary screen was then performed using flow cytometry to further characterize the positive hits identified in the primary screen. Supernatant from each of the 26 candidate clones was tested for its ability to bind four cell types: D17 (canine osteosarcoma), OSCA-8 (canine osteosarcoma), EFB (canine hemangiosarcoma), and canine PBMCs. Of the 26 clones tested, 5 lead candidates were selected for recombinant production and further characterization (9E2, 9F4, 12A2, 12F1; FIG. 3). As shown in FIG. 2, the candidate antibodies are able to label cancer cells in flow cytometry and show minimal background staining with PBMCs. The positive control used in these studies was a commercially-available, well-characterized polyclonal N-cad antibody (Abcam, Ab76057). This reagent has been reported in 38 publications, many of which use the reagent to label circulating cells undergoing epithelial-to-mesenchymal (ETM) transition. While the polyclonal N-cad did demonstrate good affinity for the cancer cells, it also exhibited non-specific binding to canine PBMCs.

All publications, patents, patent applications and accession numbers mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. An antibody that specifically binds canine N-cadherin, wherein the antibody comprises a heavy chain variable region comprising: a CDR1 sequence selected from the group consisting of SEQ ID NOs: 66-70; a CDR2 sequence selected from the group consisting of SEQ ID NOs: 71-75; and a CDR3 sequence selected from the group consisting of SEQ ID NOs: 76-80; and wherein the antibody comprises a light chain variable region comprising: a CDR1 sequence selected from the group consisting of SEQ ID NOs: 26-30; a CDR2 sequence selected from the group consisting of SEQ ID NOs: 31-35; and a CDR3 sequence selected from the group consisting of SEQ ID NOs: 36-40.

2. An antibody that specifically binds canine N-cadherin, wherein said antibody comprises a heavy chain variable region sequence selected from the group consisting of SEQ ID NO: 41-45 and sequences at least 80% identical therein and/or wherein the light chain variable region sequence selected from the group consisting of SEQ ID NO: 1-5 and sequences at least 80% identical therein.

3. The antibody of claim 2, wherein said antibody comprises a heavy chain variable region sequence selected from the group consisting of SEQ ID NO: 41-45 and sequences at least 90% identical therein and/or wherein the light chain variable region sequence selected from the group consisting of SEQ ID NO: 1-5 and sequences at least 90% identical therein.

4. The antibody of claim 2, wherein said antibody comprises a heavy chain variable region sequence selected from the group consisting of SEQ ID NO: 41-45 and sequences at least 95% identical therein and/or wherein the light chain variable region sequence selected from the group consisting of SEQ ID NO: 1-5 and sequences at least 95% identical therein.

5. A method of identifying the presence of cancer cells in a biological sample, comprising:
a) isolating and capturing circulating tumor cells (CTC) from a biological sample; and
b) detecting the presence of N-cadherin on said captured CTCs using an antibody or fragment thereof that specifically binds to canine N-cadherin.

6. The method of claim 5, wherein said sample is blood.

7. The method of claim 5, wherein said isolating and capturing comprises the use of a microfluidic chip.

8. The method of claim 5, wherein said antibody is labeled with a detectable label.

9. The method of claim 5, wherein said detecting is performed on said captured CTCs in said microfluidic chip.

10. The method of claim 5, wherein said method further comprises detecting the presence of captured white blood cells (WBCs).

11. The method of claim 10, wherein said WBSs are identified by detecting the presence of a CD45 polypeptide on said captured WBC.

12. The method of claim 5, wherein said cancer comprises one or more cancers selected from the group consisting of hemangiosarcoma, osteosarcoma, mammary cancers, mixed cancers, and carcinomas.

13. The method of claim 12, wherein said assay identifies the presence of any one of said cancers.

14. The method of claim 5, wherein said sample is obtained from a non-human subject.

15. The method of claim 14, wherein said subject is a canine subject.

16. The method of claim 5, wherein said antibody comprises a heavy chain variable region comprising: a CDR1 sequence selected from the group consisting of SEQ ID NOs: 66-70; a CDR2 sequence selected from the group consisting of SEQ ID NOs: 71-75;
and a CDR3 sequence selected from the group consisting of SEQ ID NOs: 76-80; and
wherein the antibody comprises a light chain variable region comprising: a CDR1 sequence selected from the group consisting of SEQ ID NOs: 26-30; a CDR2 sequence selected from the group consisting of SEQ ID NOs: 31-35; and a CDR3 sequence selected from the group consisting of SEQ ID NOs: 36-40.

17. The method of claim 5, wherein said antibody comprises a heavy chain variable region sequence selected from the group consisting of SEQ ID NO: 41-45 and sequences at least 80% identical therein and/or wherein the light chain variable region sequence selected from the group consisting of SEQ ID NO: 1-5 and sequences at least 80% identical therein.
